(19) United States
(12) Patent Application Publication
(10) Pub. No.: US 2019/0084907 A1
(43) Pub. Date: Mar. 21, 2019

(54) PROCESS FOR RECOVERING A METALLIC COMPONENT

(71) Applicant: SHELL OIL COMPANY, HOUSTON, TX (US)

(72) Inventors: Pieter HUIZENGA, Amsterdam (NL); Evert VAN DER HEIDE, Amsterdam (NL); Johannes Pieter HAAN, Amsterdam (NL); Michel VLAANDEREN, Amsterdam (NL)

(21) Appl. No.: 16/082,123

(22) PCT Filed: Mar. 6, 2017
(86) PCT No.: PCT/EP2017/055202

§ 371 Date: Sep. 4, 2018

(30) Foreign Application Priority Data
Mar. 7, 2016 (EP) 16159008.8

(51) Int. Cl. C07C 29/76; C07C 29/132; B01D 69/04; B01D 71/02; B01D 61/02; B01D 69/02

(52) U.S. Cl. CPC C07C 29/76; C07C 29/132; B01D 69/04; B01D 2325/02; B01D 61/027; B01D 69/02; B01D 2313/24; B01D 71/024

(57) ABSTRACT

The invention provides a process for recovering a metallic component from a process stream, said process comprising passing said process stream over a ceramic membrane comprising a selective layer with a pore size in the range of from at least 0.5 nm to at most 10 nm; applying a pressure difference across said ceramic membrane such that the pressure outside the ceramic membrane is at least 50 kPa lower than the pressure inside the ceramic membrane; and, thus, providing a permeate stream which has passed through the ceramic membrane and which is depleted in the metallic component and a retentate stream enriched in the metallic component; wherein the process stream is derived from a process for the conversion of saccharide-containing feedstock into glycols.

### FIELD OF THE INVENTION

This invention relates to a process for recovering a metallic component from a process stream and to a process for preparing glycols from a saccharide-containing feedstock.

### BACKGROUND OF THE INVENTION

Monoethylene glycol (MEG) and monopropylene glycol (MPG) are valuable materials with a multitude of commercial applications, e.g. as heat transfer media, antifreeze, and precursors to polymers such as polyethylene terephthalate (PET).

Said glycols are currently made on an industrial scale by hydrolysis of the corresponding alkylene oxides, which are the oxidation products of ethylene and propylene, generally produced from fossil fuels.

In recent years increased efforts have been focussed on reducing the reliance on fossil fuels as a primary resource for the provision of fuels and commodity chemicals. Carbohydrates and related biomass are seen as key renewable resources in the efforts to provide new fuels and alternative routes to desirable chemicals.

In particular, certain carbohydrates can be reacted with hydrogen in the presence of a catalyst system to generate polyols and sugar alcohols. Current methods for the conversion of saccharides to glycols revolve around a hydrogenation/retro-aldol process.

Reported processes generally require a first catalytic species to perform a retro-aldol reaction and a second catalytic species for hydrogenation of the products from the retro-aldol reaction.

Processes for the conversion of cellulose to products including MEG using nickel-promoted tungsten carbide catalysts are described in Angew. Chem. Int. Ed. 2008, 47, 8510-8513 and Catalysis Today 147 (2009), 77-85.

US 2011/0312487 A1 discloses a number of catalyst systems, including systems comprising tungstic acid, ammonium tungstate, ammonium metatungstate, phosphotungstic acid and ammonium paratungstate as the unsupported catalyst component in conjunction with various nickel, platinum and palladium supported catalyst components.

US 2011/03046419 A1 describes a method for producing ethylene glycol from a polyhydroxy compound such as starch, hemicellulose, glucose, sucrose, fructose and fructan in the presence of catalyst comprising a first active ingredient and a second active ingredient, the first active ingredient comprising a transition metal selected from iron, cobalt, nickel, ruthenium, rhodium, palladium, iridium, and platinum, or a mixture thereof; the second active ingredient comprising a metallic state of molybdenum and/or tungsten, or a carbide, nitride, or phosphide thereof.

WO 2015028398 describes a continuous process for the conversion of a saccharide-containing feedstock into glycols. In this process the saccharide-containing feedstock is contacted in a reactor with a catalyst composition comprising at least two active catalytic components comprising, as a first active catalyst component, one or more materials selected from transition metals from groups 8, 9 or 10 or compounds thereof, with catalytic hydrogenation capabilities; and, as a second active catalyst component, one or more materials selected from tungsten, molybdenum and compounds and complexes thereof. The second active catalyst component may be present in homogeneous form.

Regardless of the catalytic species used, homogeneous catalyst species will be present in one or more of the process streams resulting from the conversion of saccharide-containing feedstock to glycols. Such streams will include product streams, recycle streams and bleed streams.

Homogeneous catalysts are typically recycled to the reactor as components of a process stream that is withdrawn from the reactor and partially returned to the reactor. Typically, said process stream will have been subjected to separation, e.g. distillation, in order to heave removed therefrom low boiling materials including the desired product glycols. The process stream, therefore, consists mainly of heavy hydrocarbon products that are formed in the glycol production process, e.g. the stream may comprise C₃₊ sugar alcohols and carboxylic acids. A portion of this stream is removed as a bleed stream in order to prevent build-up of inerts and contaminants in the process. The bleed stream may be disposed of via flaring. This flaring typically destroys homogeneous catalysts that are present in the bleed stream, and may lead to release of metal-containing gases into the environment. Recovery of metal components from ash produced in such flaring may also be cumbersome and expensive.

The present inventors have sought to provide a process wherein metallic components (typically the homogeneous catalyst composition) may be recovered from process streams, including a bleed stream, produced in the process for the production of glycols from saccharide-containing feedstocks. Recovery of such components may enable further use of the metal and may also help to avoid release of emissions resulting from the metallic components.

### SUMMARY OF THE INVENTION

Accordingly, the present invention provides a process for recovering a metallic component from a process stream, said process comprising passing said process stream over a ceramic membrane comprising a selective layer with a pore size in the range of from at least 0.5 nm to at most 10 nm; applying a pressure difference across said ceramic membrane such that the pressure outside the ceramic membrane is at least 50 kPa lower than the pressure inside the ceramic membrane; and, thus, providing a permeate stream which has passed through the ceramic membrane and which is depleted in the metallic component and a retentate stream enriched in the metallic component; wherein the process stream is derived from a process for the conversion of saccharide-containing feedstock into glycols.

The present invention also provides a process for process for preparing glycols from a saccharide-containing feedstock comprising steps of:
i) providing a saccharide-containing feedstock in a solvent and hydrogen to a reactor system, wherein the reactor system contains at least two active catalytic compositions, said active catalyst compositions comprising, as a hydrogenation catalyst composition, one or more materials selected from transition metals from groups 8, 9 or 10 or compounds thereof, with catalytic hydrogenation capabilities; and, as a retro-aldol catalyst composition, one or more homogeneous catalysts selected from tungsten, molybdenum, lanthanum, tin or compounds or complexes thereof;
ii) withdrawing a reactor product stream from the reactor system;
iii) separating the reactor product stream into at least a glycol product stream and a hydrocarbon heavies process stream, wherein the hydrocarbon heavies process stream contains a metallic component; and
iv) passing at least a portion of the hydrocarbon heavies process stream over a ceramic membrane comprising a selective layer with a pore size in the range of from at least 0.5 nm to at most 10 nm; applying a pressure difference across said ceramic membrane such that the pressure outside the ceramic membrane is at least 50 kPa lower than the pressure inside the ceramic membrane; and, thus providing a permeate stream which has passed through the ceramic membrane and which is depleted in the metallic component and a retentate stream enriched in the metallic component.

The present invention also provides a process for preparing glycols from a saccharide-containing feedstock comprising steps of:
i) contacting said saccharide-containing feedstock in a solvent and, optionally, hydrogen with a homogeneous retro-aldol catalyst composition in a first reaction zone within a reactor system, to provide an intermediate process stream comprising at least glycolaldehyde and a metallic component in a solvent;
ii) passing at least a portion of said intermediate process stream over a ceramic membrane comprising a selective layer with a pore size in the range of from at least 0.5 nm to at most 10 nm; applying a pressure difference across said ceramic membrane such that the pressure outside the ceramic membrane is at least 50 kPa lower than the pressure inside the ceramic membrane; and, thus, providing a permeate stream which has passed through the ceramic membrane and which is depleted in the metallic component and a retentate stream enriched in the metallic component; and
iii) providing said permeate stream to a second reaction zone within the reactor system and contacting it therein with hydrogen in the presence of a hydrogenation catalyst composition to provide a product stream comprising glycols.

### BRIEF DESCRIPTION OF THE DRAWINGS

FIGS. 1 to 3 are schematic diagrams representing exemplary, but non-limiting, aspects of the process of the invention.

FIG. 4 illustrates the process carried out in the Examples.

### DETAILED DESCRIPTION OF THE INVENTION

The present inventors have surprisingly found that a ceramic membrane may be effectively used to separate metallic components from a process stream derived from a process for the conversion of saccharides into glycols. Such a process allows the separation of metallic components from process streams at temperatures at or near the reaction temperatures used in typical processes for the conversion of saccharides into glycols. This is particularly advantageous when the process stream is a stream that is to be recycled to the process or when it is to be immediately used in another process step, as it cuts out any cooling and heating steps. The metallic components may then be recycled to the process.

Said process stream comprises a metallic component that may suitably be a metallic homogeneous catalyst composition or the degradation products that can result when such a metallic catalyst composition or a heterogeneous metallic catalyst composition degrades. The metallic component in the process stream suitably comprises one or more compound, complex or elemental material comprising tungsten, molybdenum, lanthanum or tin. Preferably, the metallic component comprises one or more compound, complex or elemental material selected from those containing tungsten or molybdenum. More preferably, the metallic component comprises one or more material selected from the list consisting of tungstic acid, molybdic acid, ammonium tungstate, ammonium metatungstate, ammonium paratungstate, tungstate compounds comprising at least one Group I or II element, metatungstate compounds comprising at least one Group I or II element, paratungstate compounds comprising at least one Group I or II element, heteropoly compounds of tungsten, heteropoly compounds of molybdenum, tungsten oxides, molybdenum oxides and combinations thereof.

The process stream typically comprises from 0.1 to 20 wt % of metallic components, based upon the weight of the metal compared to the weight of the hydrocarbon product stream, preferably from 2 to 18 wt %, more preferably from 5 to 15 wt %.

The process stream is passed over a ceramic membrane. Said ceramic membrane suitably takes the form of a ceramic membrane disc or a tubular ceramic membrane disposed along the path that the process stream takes. In the embodiment that the ceramic membrane is a tubular ceramic membrane, said tubular ceramic membrane is open-ended and is preferably disposed within a pipe, such that a portion of the pipe is formed of the tubular ceramic membrane.

The ceramic membrane comprises a selective layer has a pore size in the range of from at least 0.5 nm to at most 10 nm. Preferably, the pore size is in the range of from at least 0.9 nm to at most 5 nm.

Preferably, the material of which the selective layer of the ceramic membrane is made is selected from titania, zirconia and alumina with pore sizes in this range. Said selective layer of the ceramic membrane is typically supported on one or more further layers of oxide supports, preferably alumina. The one or more further layers will suitably have larger pore sizes than the selective layer membrane itself.

A pressure difference is applied across the ceramic membrane such that the pressure outside the ceramic membrane is at least 50 kPa lower than the pressure inside the ceramic membrane, preferably at least 100 kPa lower than the pressure inside the ceramic membrane. Also preferably, the pressure outside the ceramic membrane is no more than 4 MPa lower than the pressure inside the ceramic membrane.

The permeate stream is depleted in the metallic component. Preferably, the permeate stream contains less than 50 wt %, more preferably no more than 20 wt %, even more preferably no more than 10 wt %, even more preferably no more than 5 wt %, even more preferably no more than 3 wt %, most preferably no more than 1 wt % of the metallic component present in the process stream to be treated.

The retentate stream does not pass through the ceramic membrane and is enriched in the metallic component. Preferably, the retentate stream contains more than 50 wt %, more preferably at least 80wt&, even more preferably at least 90 wt %, even more preferably at least 95 wt %, even more preferably at least 97 wt %, most preferably at least 99 wt % of the metallic component present in the process stream to be treated.

The process stream is derived from a process for the conversion of saccharide-containing feedstock into glycols. Any suitable process stream, including those set out in the description of processes for preparing glycols from a saccharide-containing feedstock below, may be used.

The present invention also provides processes for preparing glycols from a saccharide-containing feedstock. In said processes, the saccharide-containing feedstock contains one or more saccharides that are selected from the group consisting of monosaccharides, disaccharides, oligosaccharides and polysaccharides.

Saccharides, also referred to as sugars or carbohydrates, comprise monomeric, dimeric, oligomeric and polymeric aldoses, ketoses, or combinations of aldoses and ketoses, the monomeric form comprising at least one alcohol and a carbonyl function, being described by the general formula of CₙH₂ₙOₙ (n=4, 5 or 6). Typical C₄ monosaccharides comprise erythrose and threose, typical C₅ saccharide monomers include xylose and arabinose and typical C₆ sugars comprise aldoses like glucose, mannose and galactose, while a common C₆ ketose is fructose. Examples of dimeric saccharides, comprising similar or different monomeric saccharides, include sucrose, maltose and cellobiose. Saccharide oligomers are present in corn syrup. Polymeric saccharides include cellulose, starch, glycogen, hemicellulose, chitin, and mixtures thereof.

If the one or more saccharides comprise oligosaccharides or polysaccharides, it is preferable that they are subjected to pre-treatment before being fed to the process in a form that can be converted in the process of the present invention. Suitable pre-treatment methods are known in the art and one or more may be selected from the group including, but not limited to, sizing, drying, grinding, hot water treatment, steam treatment, hydrolysis, pyrolysis, thermal treatment, chemical treatment, biological treatment. However, after said pre-treatment, the starting material still comprises mainly monomeric and/or oligomeric saccharides. Said saccharides are, preferably, soluble in the reaction solvent.

In one preferred embodiment of the invention, the one or more saccharides present in the saccharide-containing feedstock used in the process of the invention, after any pre-treatment, comprise saccharides selected from starch and/or hydrolysed starch. Hydrolysed starch comprises glucose, sucrose, maltose and oligomeric forms of glucose.

In another preferred embodiment of the invention, the one or more saccharides in the saccharide-containing feedstock comprise cellulose, hemi-cellulose, saccharides derived from lignocellulose, and/or sugars derived therefrom. In this embodiment, the one or more saccharides are preferably derived from softwood.

The one or more saccharides in the saccharide-containing feedstock may be derived from grains such as corn, wheat, millet, oats, rye, sorghum, barley or buckwheat, from rice, from pulses such as soybean, pea, chickpea or lentil, from bananas and/or from root vegetables such as potato, yam, sweet potato, cassava and sugar beet, or any combinations thereof. A preferred source of saccharide-containing feedstock is corn.

The one or more saccharides are suitably present as a solution, a suspension or a slurry in the solvent.

The process of the present invention is carried out in the presence of a solvent. The solvent may be water or a C₁ to C₆ alcohol or polyalcohol (including sugar alcohols), ethers, and other suitable organic compounds or mixtures thereof. Preferred C₁ to C₆ alcohols include methanol, ethanol, 1-propanol and iso-propanol. Polyalcohols of use include glycols, particularly products of the hydrogenation/ retro-aldol reaction, glycerol, erythritol, threitol, sorbitol and mixtures thereof. Preferably, the solvent comprises water.

Suitably the ratio of saccharide-containing feedstock and solvent are adjusted such that the feedstock to the reactor system contains solvent:saccharide in a ratio of between 1:1 and 5:1.

Hydrogen is required for the part of the process comprising contacting a stream with the hydrogenation catalyst composition. It may be supplied at the start of the reactor system or only to the part of the reactor system in which hydrogenation occurs, if this is a distinct part of the system, for example to the second reactor zone. The hydrogen pressure is suitably greater than 10 bar, preferably greater than 70 bar and most preferably around 100 bar. The amount of hydrogen consumed will depend upon the amount of saccharide that is provided (1 mole of glucose will react with 3 moles of hydrogen).

Within the reactor system, there may be one or more reaction zones, within which different reactions are prevalent. For example, retro-aldol reactions may be the dominant reaction in one reaction zone and hydrogenation reactions may predominate in a further reaction zone. Each reaction zone may be a distinct part of a single reactor or each reaction zone may comprise an individual reactor.

Each reaction zone may be operated with a different temperature, pressure and catalyst make-up.

Two active catalytic compositions are used in the processes of the present invention. These active catalyst compositions comprise, as a hydrogenation catalyst composition, one or more materials selected from transition metals from groups 8, 9 or 10 or compounds thereof, with catalytic hydrogenation capabilities; and, as a retro-aldol catalyst composition, one or more homogeneous catalysts selected from tungsten, molybdenum, lanthanum, tin or compounds or complexes thereof. The retro-aldol catalyst selectively cuts the saccharide molecules into smaller components.

The hydrogenation catalyst composition is suitably heterogeneous with respect to the reaction mixture. When hydrogenation catalyst composition is heterogeneous there will not be significant quantities of metal from said catalyst composition in any process stream, although it is possible that there might be very low levels of metal (e.g. up to 10 ppm) that have leached from the heterogeneous catalyst that are found in any process stream.

The retro-aldol catalyst composition is a homogeneous catalyst so the hydrocarbon product stream will contain one or more metallic components that are either said homogeneous catalyst composition or degradation products resulting from the homogeneous retro-aldol catalyst composition.

Suitably, the hydrogenation catalyst composition comprises one or more of the group of metals selected from iron, cobalt, nickel, ruthenium, rhodium, palladium, iridium and platinum. This metal may be present in the elemental form or as a compound. A preferred catalyst is Raney nickel. Another possible catalyst is ruthenium dispersed on carbon.

The retro-aldol catalyst composition preferably comprises one or more homogeneous catalysts selected from tungsten or molybdenum, or compounds or complexes thereof. Most preferably, the second active catalyst comprises one or more material selected from the list consisting of tungstic acid, molybdic acid, ammonium tungstate, ammonium metatungstate, ammonium paratungstate, tungstate compounds comprising at least one Group I or II element, metatungstate compounds comprising at least one Group I or II element, paratungstate compounds comprising at least one Group I or II element, heteropoly compounds of tungsten, heteropoly compounds of molybdenum, tungsten oxides, molybdenum oxides and combinations thereof.

The temperature within the reactor system is suitably at least 130° C., preferably at least 150° C., more preferably at least 170° C., most preferably at least 190° C. The temperature within the reactor system is suitably at most 300° C., preferably at most 280° C., more preferably at most 270° C., even more preferably at most 250° C. Preferably, the reactor system is heated to a temperature within these limits before addition of any starting material and is maintained at such a temperature as the reaction proceeds.

The pressure in the reactor system is suitably at least 1 MPa, preferably at least 2 MPa, more preferably at least 3 MPa. The pressure in the reactor system is suitably at most 15 MPa, preferably at most 12 MPa, more preferably at most 10 MPa, most preferably at most 8 MPa. Preferably, the reactor system is pressurised to a pressure within these limits by addition of hydrogen before addition of any saccharide-containing feedstock or solvent and is maintained at such a pressure as the reaction proceeds through on-going addition of hydrogen.

When the reactor system includes more than one reaction zone and /or more than one reactor, the temperature and pressure in each reaction zone and/or reactor may be varied independently.

The process takes place, at least partly, in the presence of hydrogen. Preferably, the process takes place in the absence of air or oxygen. In order to achieve this, it is preferable that the atmosphere in the reactor be evacuated and replaced an inert gas, such as nitrogen, and then, where relevant, with hydrogen repeatedly, after loading of any initial reactor system contents, before the reaction starts.

Suitable reactors for use in the reactor system include stirred tank reactors, slurry reactors, ebullated bed reactors, jet flow reactors, mechanically agitated reactors, bubble columns, such as slurry bubble columns and external recycle loop reactors. The use of these reactors allows dilution of the reaction feedstock and intermediates to an extent that provides high degrees of selectivity to the desired glycol product (mainly ethylene and propylene glycols), such as by effective back-mixing.

The residence time in the reactor system is suitably at least 1 minute, preferably at least 2 minutes, more preferably at least 5 minutes. Suitably the residence time in the reactor system is no more than 5 hours, preferably no more than 2 hours, more preferably no more than 1 hour.

In embodiments of the invention wherein the process takes place in more than one reaction zone, this residence time may be split equally or disproportionally between the two or more reaction zones.

In embodiments wherein a reactor product stream is withdrawn from the reactor system, typically this stream contains solvent, hydrocarbons and homogeneous catalyst materials. The reactor product stream may then be separated into at least a glycol product stream and a hydrocarbon heavies process stream. Preferably, the reactor product stream is additionally separated into a light hydrocarbon stream and water. In a preferred separation step, the light hydrocarbon stream is first separated from the reactor product stream and then the water is removed by distillation. The glycol product stream is then separated from the hydrocarbon heavies process stream by distillation (the hydrocarbon heavies process stream is the bottom product from this distillation).

Said glycol product stream comprises as least one of monoethylene glycol (MEG), monopropylene glycol (MPG) and 1,2-butanediol (1,2-BDO). The different glycols may be collected as separate streams or as one combined stream.

A hydrocarbon heavies process stream is separated from the reactor product stream, and is, preferably, at least partially recycled back to the reactor, either directly or indirectly. The hydrocarbon heavies process stream typically contains heavy hydrocarbons and a metallic component comprising the second active catalyst component. The recycling of this stream enables reuse of the homogeneous second active catalyst component.

In one embodiment of the invention at least a portion of the hydrocarbon heavies process stream is passed over a ceramic membrane comprising a selective layer with a pore size in the range of from at least 0.5 nm to at most 10 nm and a pressure difference is applied across said ceramic membrane such that the pressure outside the ceramic membrane is at least 50 kPa lower than the pressure inside the ceramic membrane in order to provide a permeate stream which has passed through the ceramic membrane and which is depleted in the metallic component and a retentate stream enriched in the metallic component.

In one preferred embodiment of the invention, all or substantially all of the hydrocarbon heavies process stream is passed over the ceramic membrane. In this embodiment, the permeate stream comprises in the range of from 1 to 20 wt % and preferably around 10 wt % of the hydrocarbon heavies process stream. This permeate stream can then be treated as a bleed stream and removed from the process. The retentate stream may then be recycled to the reactor system.

In another preferred embodiment of the invention, a portion of the hydrocarbon heavies process stream is separated as a bleed stream and it is this bleed stream that is passed over the ceramic membrane. Suitably from 1 to 20 wt % and preferably around 10 wt % of the hydrocarbon heavies stream is separated to provide the bleed stream. In this embodiment, the permeate stream comprises in the range of from 50 to 95 wt % of the bleed stream. This permeate stream can then be removed from the process. Optionally, the retentate stream may then be recycled to the reactor system.

In the embodiment of the invention wherein the reactor system comprises two reaction zones and the intermediate process stream from the first reaction zone is passed over a ceramic membrane, the retentate stream may be recycled to the first reaction zone for re-use of catalytic material contained therein.

### DETAILED DESCRIPTION OF THE DRAWINGS

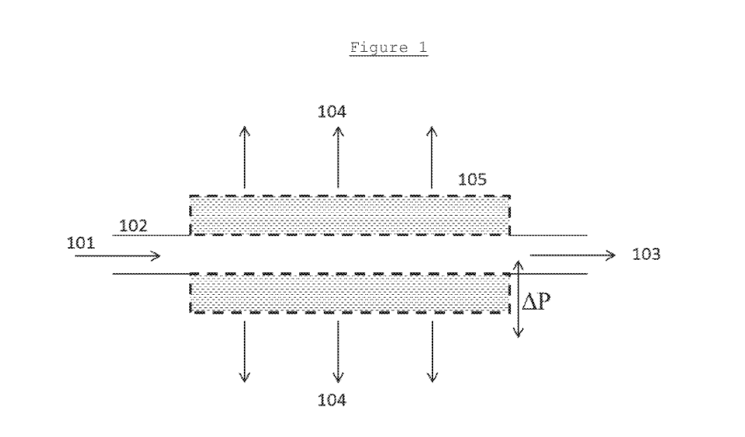

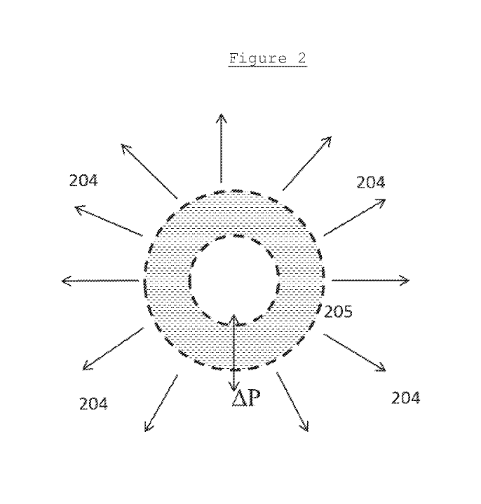

In these Figures, the first digit of each reference number refers to the Figure number (i.e. 1XX for FIGS. 1 and 2XX for FIG. 2). The remaining digits refer to the individual features and the same features are provided with the same number in each Figure. Therefore, the same feature is numbered 104 in FIGS. 1 and 204 in FIG. 2.

FIG. 1 illustrates a non-limiting, embodiment of the present invention. A process stream 101, derived from a process for the conversion of saccharide-containing feedstock into glycols and containing a metallic component, is passed through a pipe 102 and along the inside of a ceramic membrane 105 in the form of a tubular ceramic membrane. A pressure difference (AP) is applied across said tubular ceramic membrane. A permeate stream 104 which has passed through the ceramic membrane and which is depleted in the metallic component is provided. A retentate stream 103 enriched in the metallic component is also provided.

The same embodiment is shown in cross-section in FIG. 2.

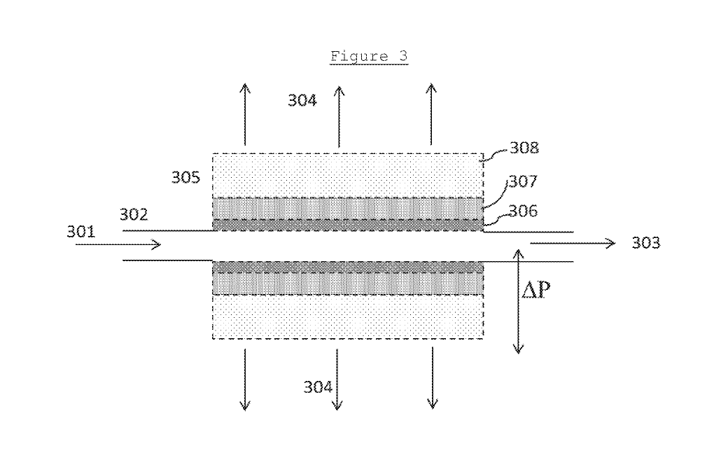

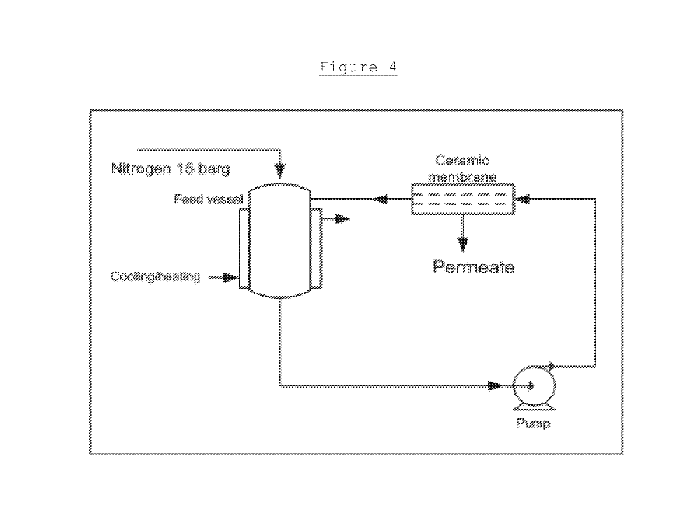

FIG. 3 illustrates a ceramic membrane 305, in the form of a tubular ceramic membrane, in which the selective layer 306 of the ceramic membrane 305 is supported on further layers 307 and 308 of oxide supports, preferably alumina. The further layers 307 and 308 will suitably have larger pore sizes than the selective layer 306 of the membrane.

The invention is further illustrated by the following Examples.

### EXAMPLES

Membrane testing was carried out in a small laboratory unit with a 500 ml feed vessel. A cross-flow of 801/hr across the ceramic membrane surface of 0.00187 m² is maintained by means of a pump.

The feed vessel is heated by means of an external oil bath and by proper insulation the desired temperature can be achieved. The unit can be pressurized at the feed side of the membrane by means of 15 barg (16 bar absolute) nitrogen. The mass of the collected permeate is recorded against time and accordingly the permeate mass flow in kg/hr is calculated.

The Trans Membrane Pressure (TMP) is the driving force for transport through the pores of the membrane and is defined as the average pressure difference between the Feed/Retentate side and the Permeate side of the membrane.

TMP=(Pin+Pout)/2−Ppermeate

Pin=Pressure at the membrane entrance (feed in)

Pout=Pressure at the membrane outlet (retentate out)

Ppermeate=Pressure at the membrane permeate side

The membrane flux (kg.m⁻².hr⁻¹) is calculated from the measured permeate flow in kg/hr divided by the used membrane area. The membrane permeability (kg.m⁻².hr⁻¹.bar⁻¹) is defined as the flux divided by the TMP. The rejection is a measure for a component (Tungsten) which does not pass the membrane and is retained by the membrane. The rejection is calculated from the Tungsten concentrations in the respective permeate and retentate streams, i.e. the Tungsten concentrations in the final total permeate and total retentate volumes.

Rejection=(1-[Wpermeate]/[Wretentate])*100%

[Wpermeate]=Tungsten concentration in total permeate (mg/kg)

[Wretentate]=Tungsten concentration in total retentate (mg/kg)

The permeate recovery relates the amount of permeate collected and the amount of feed used in the experiment.

Permeate recovery=((kg of permeate collected)/(kg of feed))*100%.

### Example 1

In 400 grams of a 1:1 (weight ratio) glycerol/water mixture 3.38 gram of sodium metatungstate.monohydrate (Na₆W₁₂O₉.H₂O) was dissolved. The feed vessel of the membrane unit was filled with 393.8 gram of this solution. A tubular 5 nm pore size membrane, Titania selective layer (ex Inopor, Germany) was installed in the cross-flow unit. The circulation pump was started and when the liquid reached a temperature of 90° C. a Trans Membrane Pressure of 15.2 bar was applied by means of pressurizing the feed side of the system with nitrogen gas. During the experiment the temperature was increased to 94° C. In approximately 3 hours, 212.9 gram of permeate was collected and after cooling down of the unit, 160.7 gram retentate could be withdrawn. From this a mass loss of 20.2 gram was calculated. A permeate mass flow of 0.070 kg/h was calculated. A summary of the results is shown in Table 1.

### Example 2

In 404 grams of a 1:1 (weight ratio) glycerol/water mixture 3.38 gram of sodium metatungstate.monohydrate (Na₆W₁₂O₉.H₂O) was dissolved. The feed vessel of the membrane unit was filled with 394.7 gram of this solution. A tubular 3 nm pore size membrane, Zirconia selective layer (ex Inopor, Germany) was installed in the cross-flow unit. The circulation pump was started and when the liquid reached a temperature of 90 ° C. a Trans Membrane Pressure of 15.2 bar was applied by means of pressurizing the feed side of the system with nitrogen gas. During experiment the temperature increased to 93° C. In approximately 2 hrs 307.9 gram of permeate was collected and after cooling down of the unit, 59.9 gram retentate could be withdrawn. From this a mass loss of 26.9 gram was calculated. A permeate mass flow of 0.158 kg/hr was calculated. A summary of the results is shown in Table 1.

TABLE 1

| | | Example 1 | Example 2 |
|---|---|---|---|
| Membrane | | 5 nm TiO₂ | 3 nm ZrO₂ |
| | | (ex Inopor) | (ex Inopor) |
| Temperature | ° C. | 90-94 | 90-93 |
| Trans Membrane | bar | 15.2 | 15.2 |
| Pressure | | | |
| Polar liquid:glycerol/water | w:w | 1:1 | 1:1 |
| Membrane area | m² | 0.00187 | 0.00187 |
| Membrane flux | kg · m⁻² · hr⁻¹ | 37.4 | 84.5 |
| Permeability | kg · m⁻² · | 2.5 | 5.6 |
| | hr⁻¹ · bar⁻¹ | | |
| Permeate recovery | % wt. | 54 | 78 |
| W content, feed | mg/kg | 6197 | 6065 |
| W content, retentate | mg/kg | 7896 | 26186 |
| W content, permeate | mg/kg | 4778 | 810 |
| W rejection | % wt. | 39.5 | 96.9 |
| W in permeate/W in | % wt. | 41.6 | 10.4 |
| Retentate (mass ratio) | | | |

The examples indicate metal recovery using both membranes. However, there is a clear difference between the two membranes under comparable conditions. The 3 nm pores size Zirconia membrane is much more selective in rejecting the Tungsten containing molecule. In this case a high rejection of Tungsten is obtained: 96.9%.

1. A process for recovering a metallic component from a process stream, said process comprising passing said process stream over a ceramic membrane comprising a selective layer with a pore size in the range of from at least 0.5 nm to at most 10 nm;
applying a pressure difference across said ceramic membrane such that the pressure outside the ceramic membrane is at least 50 kPa lower than the pressure inside the ceramic membrane; and, thus, providing a permeate stream which has passed through the ceramic membrane and which is depleted in the metallic component and a retentate stream enriched in the metallic component; wherein the process stream is derived from a process for the conversion of saccharide-containing feedstock into glycols.

2. A process for preparing glycols from a saccharide-containing feedstock comprising steps of:
i) providing a saccharide-containing feedstock in a solvent and hydrogen to a reactor system, wherein the reactor system contains at least two active catalytic compositions, said active catalyst compositions comprising, as a hydrogenation catalyst composition, one or more materials selected from transition metals from groups 8, 9 or 10 or compounds thereof, with catalytic hydrogenation capabilities; and, as a retro-aldol catalyst composition, one or more homogeneous catalysts selected from tungsten, molybdenum, lanthanum, tin or compounds or complexes thereof;
ii) withdrawing a reactor product stream from the reactor system;
iii) separating the reactor product stream into at least a glycol product stream and a hydrocarbon heavies process stream, wherein the hydrocarbon heavies process stream contains a metallic component; and
iv) passing at least a portion of the hydrocarbon heavies process stream over a ceramic membrane comprising a selective layer with a pore size in the range of from at least 0.5 nm to at most 10 nm; applying a pressure difference across said ceramic membrane such that the pressure outside the ceramic membrane is at least 50 kPa lower than the pressure inside the ceramic membrane; and, thus providing a permeate stream which has passed through the ceramic membrane and which is depleted in the metallic component and a retentate stream enriched in the metallic component.

3. A process for process for preparing glycols from a saccharide-containing feedstock comprising steps of:
i) contacting said saccharide-containing feedstock in a solvent and, optionally, hydrogen with a homogeneous retro-aldol catalyst composition in a first reaction zone within a reactor system, to provide an intermediate process stream comprising at least glycolaldehyde and a metallic component in a solvent;
ii) passing at least a portion of said intermediate process stream over a ceramic membrane comprising a selective layer with a pore size in the range of from at least 0.5 nm to at most 10 nm; applying a pressure difference across said ceramic membrane such that the pressure outside the ceramic membrane is at least 50 kPa lower than the pressure inside the ceramic membrane; and, thus, providing a permeate stream which has passed through the ceramic membrane and which is depleted in the metallic component and a retentate stream enriched in the metallic component; and
iii) providing said permeate stream to a second reaction zone within the reactor system and contacting it therein with hydrogen in the presence of a hydrogenation catalyst composition to provide a product stream comprising glycols.

4. The process as claimed in claim 2, wherein all or substantially all of the hydrocarbon heavies process stream is passed over the ceramic membrane and the permeate stream comprises in the range of from 1 to 20 wt % of the hydrocarbon heavies process stream.

5. The process as claimed in claim 2, wherein from 1 to 20 wt % of the hydrocarbon heavies process stream is passed over the ceramic membrane having been separated as a bleed stream and the permeate stream comprises in the range of from 50 to 95 wt % of said bleed stream.

6. The process as claimed in claim 2, wherein the retentate stream is recycled to the reactor system.

7. The process as claimed in claim 1, wherein the ceramic membrane is in the form of a tubular ceramic membrane or a ceramic membrane disc.

8. The process as claimed in claim 1, wherein the selective layer of the ceramic membrane is made from a material selected from titania, zirconia, alumina and mixtures thereof.

9. The process as claimed in claim 1, wherein the selective layer of the ceramic membrane is supported on one or more further layers of oxide support having larger pore sizes than the selective layer.

10. The process as claimed in claim 1, wherein the permeate stream contains no more than 3 wt % of the metallic component present in the process stream.
